(19) United States
(12) Patent Application Publication
(10) Pub. No.: US 2017/0086797 A1
(43) Pub. Date: Mar. 30, 2017

(54) METHODS AND SYSTEMS FOR MANAGING DISTRIBUTION OF PROTECTED INFORMATION ON A MEDICAL DISPLAY

(71) Applicant: General Electric Company, Schenectady, NY (US)

(72) Inventors: Menachem Nahi Halmann, Waukesha, WI (US); Mark Steven Urness, Waukesha, WI (US)

(21) Appl. No.: 14/866,252

(22) Filed: Sep. 25, 2015

(51) Int. Cl. A61B 8/00; A61B 8/08; G06F 21/62

(52) U.S. Cl. CPC A61B 8/56; G06F 21/6245; A61B 8/465; A61B 8/5207

(57) ABSTRACT

Methods and systems managing protected health information (PHI) on a medical display are provided. The systems and methods detect a plurality of communication links between a medical device and a plurality of remote system and display the medical device and the plurality of remote system as corresponding graphical icons on a display. The systems and methods further determine encryption levels for the plurality of communication links and display connection graphics representing the plurality of communication links. Each connection graphic is positioned between the medical device and one of the remote systems having a visual feature corresponding to an encryption level of a communication link between the medical device and the one of the remote systems.

### BACKGROUND OF THE INVENTION

Embodiments described herein generally relate to managing protected health information using an object-oriented interface of a medical display.

An ultrasound imaging system typically includes an ultrasound probe that is applied to a patient's body and a workstation or device that is operably coupled to the probe. The probe may be controlled by an operator of the system and is configured to transmit and receive ultrasound signals that are processed into an ultrasound image by the workstation or device. The workstation or device may show the ultrasound images through a display device.

Before each imaging session, an operator typically sets up the ultrasound system for the particular type of scan to be performed. In a typical process, an operator accesses protected health information (PHI) of the patient, for example, from a Digital Imaging and Communications in Medicine (DICOM) worklist to select a patient for the upcoming ultrasound scan to be performed. The selection of the patient from the DICOM worklist typically populates the data fields on the screen of the ultrasound system with patient demographic information. After the scan is performed, the PHI can be transferred to an external system such as an external flash drive, a billing system, or a patient archive communication system (PACS).

PHI includes confidential patient information. The use and disclosure of information within the PHI is regulated, for example, based on the Health Insurance Portability and Accountability Act and enforced by the U.S. Department of Health and Human Services (HHS). If PHI from the ultrasound imaging system or the other medical devices are stolen and/or made public to a third party, the HHS may issue fines for each unencrypted PHI. Thus, users of the ultrasound imaging system or the other medical devices and healthcare administrators need to know where the PHI is stored or located and how the PHI is transmitted.

Conventionally, the workflow or management of PHI between the ultrasound imaging system and external servers (e.g., the PACS) must be entered or set up manually by an expert technician. For example, an onsite field engineer, hospital biomed, online center personnel, and/or the like will manually enter port numbers (e.g., TCP ports, UDP ports), interface ports (e.g., USB), and/or the like into the ultrasound system and stored in text or specification files.

However, in clinical settings the ultrasound imaging system may be shared by multiple departments or an emergency room, which may have to perform multiple different exams. Moreover, different department, clinics or medical facilities may have different workflows associated with the ultrasound system. Further, many users and healthcare administrators don't have the technical expertise and/or time to analyze the various test or specification files to determine the workflow of PHI for the ultrasound imaging system. As a result, users and healthcare administrators may be unable to know the location and state of PHI accessed and/or generated by the ultrasound imaging system. Thus, increasing the risk of lost and/or third party access to PHI.

### BRIEF DESCRIPTION OF THE INVENTION

In one embodiment, a method for managing protected health information is provided. The method may include detecting a plurality of communication links between a medical device and a plurality of remote systems. The method may include displaying the medical device and the remote system as corresponding graphical icons on a display, and determine encryption levels for the plurality of communication links. The method may further include displaying connection graphics representing the plurality of communication links. Each connection graphic is positioned between the medical device and one of the remote systems, and have a visual feature corresponding to an encryption level of a communication link between the medical device and the one of the remote systems.

In another embodiment, an ultrasound imaging system is provided. The ultrasound imaging system may include a display, and a communication interface circuit configured to establish a first communication link for receiving protected health information (PHI) from a first remote system and a second communication link for transmitting updated PHI to a second remote system. The ultrasound imaging system may also include a memory configured to store programmed instructions and one or more processors to execute the programmed instructions by performing one or more operations. The one or more operations may include displaying on the display graphical icons corresponding to an ultrasound imaging system, the first remote system, and the second remote system, determining encryption levels of the first communication link and the second communication link, and displaying the first connection graphic representing the first communication link and a second connection graphic representing the second communication link on the display. The first connection graphic including at least one first visual feature corresponding to a first encryption level of the first communication link. The second connection graphic including at least one second visual feature corresponding to a second encryption level of the second communication link.

In another embodiment, a tangible and non-transitory computer readable medium comprising one or more programmed instructions configured to direct one or more processors to perform one or more operations. The one or more processors may be directed to detect a plurality of communication links between a medical device and a plurality of not remote systems, and display the medical device and each remote system as corresponding graphical icons on a display, determine encryption levels of the plurality of communication links, and display connection graphics representing the plurality of communication links. Each connection graphic is positioned between the medical device and one of the remote systems, and include a visual feature corresponding to an encryption level of the communication link between the medical device and the one of the remote systems.

### BRIEF DESCRIPTION OF THE DRAWINGS

FIG. 1 is a diagram illustrating a medical setting in which various embodiments may be implemented.

FIG. 2 is a schematic block diagram of an ultrasound imaging system in accordance with an embodiment.

FIG. 3 illustrates a screenshot of a protected health information workflow shown on a medical display in accordance with an embodiment.

FIG. 4 is a flowchart of a method for managing protected health information for a medical device in accordance with various embodiments.

FIG. 5 illustrates a screenshot of a protected health information workflow shown on a medical display in accordance with an embodiment.

FIG. 6 illustrates a screenshot of a protected health information workflow shown on a medical display in accordance with an embodiment

FIG. 7 illustrates a 3D capable miniaturized ultrasound system having a probe that may be configured to acquire 3D ultrasonic data or multi-plane ultrasonic data.

FIG. 8 illustrates a hand carried or pocket-sized ultrasound imaging system wherein the display and user interface form a single unit.

FIG. 9 illustrates an ultrasound imaging system provided on a movable base.

### DETAILED DESCRIPTION OF THE INVENTION

The following detailed description of certain embodiments will be better understood when read in conjunction with the appended drawings. To the extent that the figures illustrate diagrams of the functional modules of various embodiments, the functional blocks are not necessarily indicative of the division between hardware circuitry. Thus, for example, one or more of the functional blocks (e.g., processors or memories) may be implemented in a single piece of hardware (e.g., a general purpose signal processor or a block of random access memory, hard disk, or the like). Similarly, the programs may be stand-alone programs, may be incorporated as subroutines in an operating system, may be functions in an installed software package, and the like. It should be understood that the various embodiments are not limited to the arrangements and instrumentality shown in the drawings.

As used herein, an element or step recited in the singular and proceeded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising” or “having” an element or a plurality of elements having a particular property may include additional elements not having that property.

Various embodiments provide systems and methods for managing protected health information (PHI) by controlling, displaying, and reporting through an information system using an object-oriented methodology. For example, embodiments herein provide non-technical users to manage the flow and location of PHI on medical devices by providing a graphical, non-technical view of where PHI is stored, where PHI is at risk, and where PHI is protected. In various embodiments, a technical configuration is integrated with a graphical, object-oriented interface to display and/or adjust a PHI workflow. Optionally, a PHI workflow may be distributed to other medical devices in the organization and/or individually link the data flows to individual user accounts providing user specific data handling options. For example, a medical student may have a different workflow than an attending physician. In variously embodiments, warnings and/or reports may be generated tracking when PHI is transferred from a medical device, such as an ultrasound imaging system.

A technical effect of at least one embodiment is a more efficient verification of the PHI workflow. A technical effect of at least one embodiment increases the efficiency for distributing a PHI workflow to more than one medical device.

It should be noted that although the various embodiments may be described in connection with an ultrasound imaging system, the methods and systems are not limited to ultrasound imaging or a particular configuration thereof. The various embodiments may be implemented in connection with different types of diagnostic medical imaging systems, including, for example, x-ray imaging systems, magnetic resonance imaging (MRI) systems, computed-tomography (CT) imaging systems, positron emission tomography (PET) imaging systems, or combined imaging systems, among others.

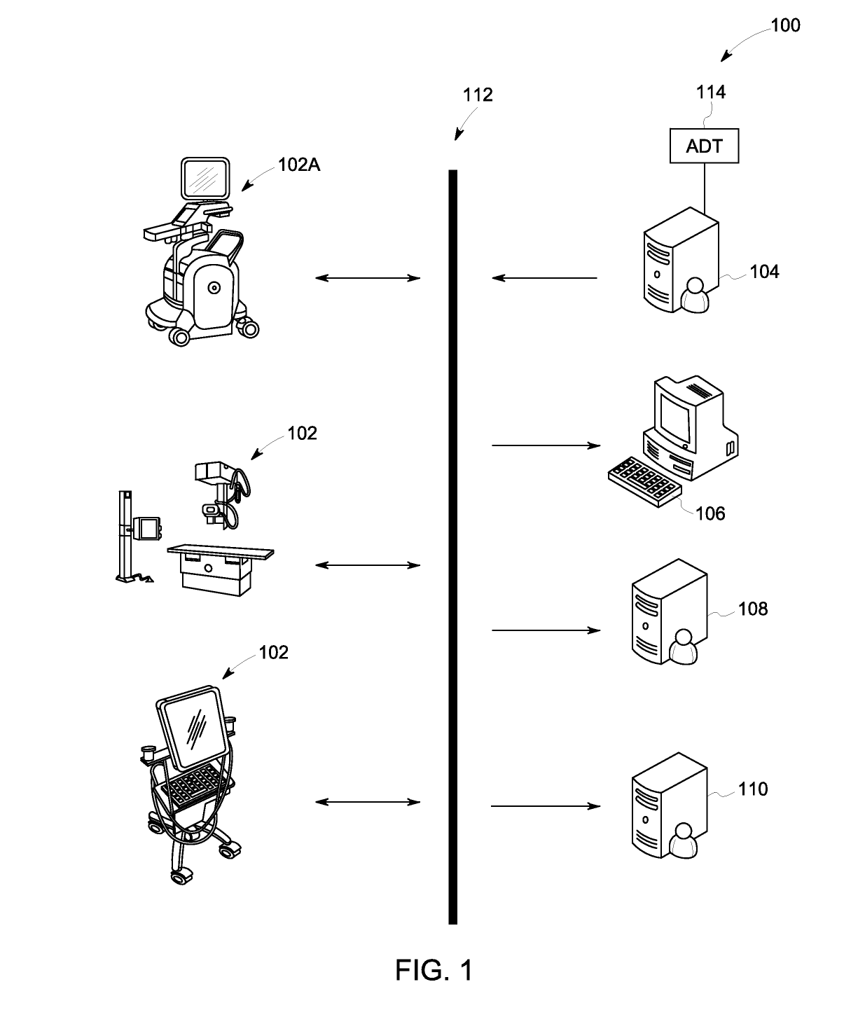

FIG. 1 illustrates a medical network 100 in which various embodiments may be implemented. The medical network 100 may correspond to multiples departments within a medical facility or multiple locations at different medical facilities. In the illustrated embodiment, a plurality of medical devices 102 are operable to perform one or more medical examinations or scans. For example, the medical devices 102 may include ultrasound imaging systems or devices (e.g., the medical device 102A), nuclear medicine imaging devices (e.g., Positron Emission Tomography (PET) or Single Photon Emission Computed Tomography (SPECT) imaging systems), Magnetic Resonance (MR) imaging devices, Computed Tomography (CT) imaging devices, and/or x-ray imaging devices, among others. It should be noted that although a description of the operation of an ultrasound imaging system in accordance with various embodiments is provided herein, the various embodiments may be implemented in connection with different ones of the medical devices 102 or other medical devices.

The medical devices 102 are communicatively coupled to one or more remote systems (e.g., a patient reference system 104, a monitoring system 106, a billing system 108, a picture archive communication system 110) via one or more communication links 112. The remote systems may be a stand-alone computing device, a server, a peripheral device, and/or other processing machines. It should be noted in other embodiments the medical network 100 may include additional remote systems or less remote systems than illustrated in FIG. 1.

The patient reference system 104 accesses and/or stores a database in a memory device that includes protected health information (PHI) such as a list of patients (including demographic information) and the corresponding type of scan or examination to be performed by one or more of the medical devices 102. For example, the database and/or PHI may correspond to Digital Imaging and Communications in Medicine (DICOM) worklists, which includes a list of examinations for one or more medical devices and associated information that may be communicated using the DICOM standard. In another example, the PHI may correspond to Electronic Medical Records (EMR).

In various embodiments, the PHI may include a name of a patient, examination information, a geographical identifier of the patient (e.g., home address, zip code, state), birth data, phone number, insurance information, patient medical history, patient characteristics (e.g., weight, age, race), and/or the like. Additionally or alternatively, the PHI may include individually identifiable health information identified by the Health Insurance Portability and Accountability Act (HIPAA) and/or the U.S. Department of Health and Human Services (HHS).

In various embodiments, the PHI is generated from information received from an Admissions/Discharge/Transfer (ADT) system 114. For example, information input into the ADT system 114, such as patient information and scheduling of examinations or scans is used to generate the PHI (e.g., DICOM worklist). The PHI may include the date, time, name, patient ID and other information that is acquired from the ADT system 114. Additionally, the PHI may include the type of examination or scan to be performed by the one or more medical devices 102 (e.g., cardiac ultrasound scan, stress echo study or emergency department exam). Thus, in various embodiments the PHI include information that may be communicated to the medical devices 102 to allow a determination of the patient and type of examination or scan to be performed by the medical device 102.

The medical devices 102 and the patient reference system 104 may communicate over the one or more communication links 112, which may be any suitable wired and/or wireless connection. For example, the various components may be connected in a local area network (LAN) or similar type of arrangement. Additionally, the medical devices 102 may be coupled to the patient reference system 104 through the same or different communication links 112, which may use the same or different communication protocols for transferring data there between. In various embodiments, the PHI is communicated from the patient reference system 104 to one or more of the medical devices 102. In various embodiments, the PHI includes patient information (e.g., used to identify the patient) and a description of the examination, scan or study to be performed using the particular medical device 102. Accordingly, in various embodiments, different PHI may be received by each of the medical devices 102.

The billing system 108, and the picture archive communication system (PACS) 110 may receive and/or store updated PHI that includes data (e.g., medical images, timestamps, diagnostics) acquired by one or more of the medical devices 102 based on the scans described in the PHI. The billing system 108, and/or the PACS 110 may receive the update PHI over the one or more communication links 112 from the medical devices 102. In various embodiments, a clinician such as a nurse and/or doctor may use the PACS 110 to evaluate and/or diagnose the patient using the updated PHI stored on the PACS 110. In another example, the billing system 108 may determine charges to the patient based on the scans completed by the one or more medical devices 102.

The PACS 110 may store the medical images (e.g., x-rays, ultrasound images, three-dimensional renderings) as, for example, imaged in a database or registry corresponding to an EMR. In some examples, the medical images are stored in the PACS 110 using a DICOM format. Additionally or alternatively, the medical images may be burned or embed portions of the corresponding PHI into the medical image. For example, the medical image may include a date of the scan, name of the patient, identification number of the patient and/or medical device 102, and/or the like that was included in the PHI.

Additionally or alternatively, the one or more of the communication links 112 may be encrypted between the one or more remote systems (e.g., the patient reference system 104, the billing system 108, the PACS 110) and the medical devices 102. For example, the content of the PHI may be encrypted by the patient reference system 104 using an Advanced Encryption Standard (AES) algorithm, an RSA algorithm standard (e.g., RSA-1024, RSA-2048), Secure Hash Algorithm (e.g., SHA-1, SHA-256, SHA-384, SHA-2), and/or the like. In another example, a password based encryption may be used such as a PKCS series. Additionally or alternatively, the encryption may be based on a DICOM encryption standard, for example, as described in ISO standard 12052:2006 and NEMA standard PS3.

The monitoring system 106 may monitor PHI transmissions of the medical devices 102 within the medical network 100 allowing a user to determine locations of PHI within the medical network 100. For example, the monitoring system 106 may include a PHI transaction report for the medical network 100. The PHI transaction report may be a collection of transmission information each corresponding to information of updated PHIs that are transmitted from the medical devices 102 to one or more of the remote systems within the medical network 100. The PHI transaction report may include a portion of the PHI, such as a patient name and/or scanning information corresponding to the updated PHI, a time stamp of the transmission, encryption information, and the intended remote system receiving the updated PHI. In various embodiments, the medical devices 102 may transmit the PHI transaction report to the monitoring system 106 periodically at a set time interval or automatically after a transmission of the updated PHI. The monitoring system 106 may combine the various PHI transaction reports received from the medical devices 102 into a stored PHI transaction report for the medical network 100.

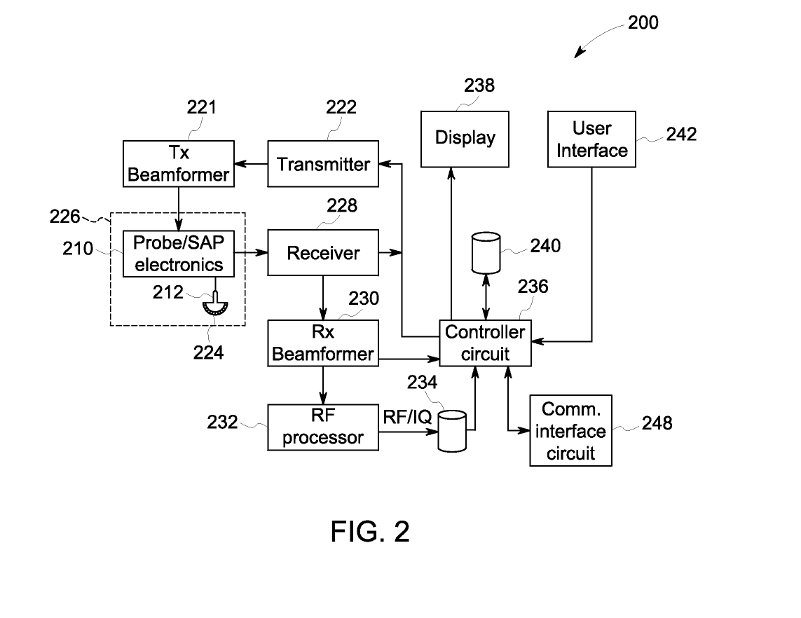

In connection with FIG. 2, the medical devices 102 may include a user interface 242 that allow a user or operator to interface with the medical device 102 to control and/or display a workflow of the PHI. The PHI workflow may correspond to a transfer and/or transmission path of the PHI with respect to the one or more communication links 112 between the remote systems and the medical devices 102. For example, the PHI workflow may indicate where (e.g., the remote system) the medical device 102 receives the PHI, and where (e.g., the remote system) the medical device 102 transmits the updated PHI.

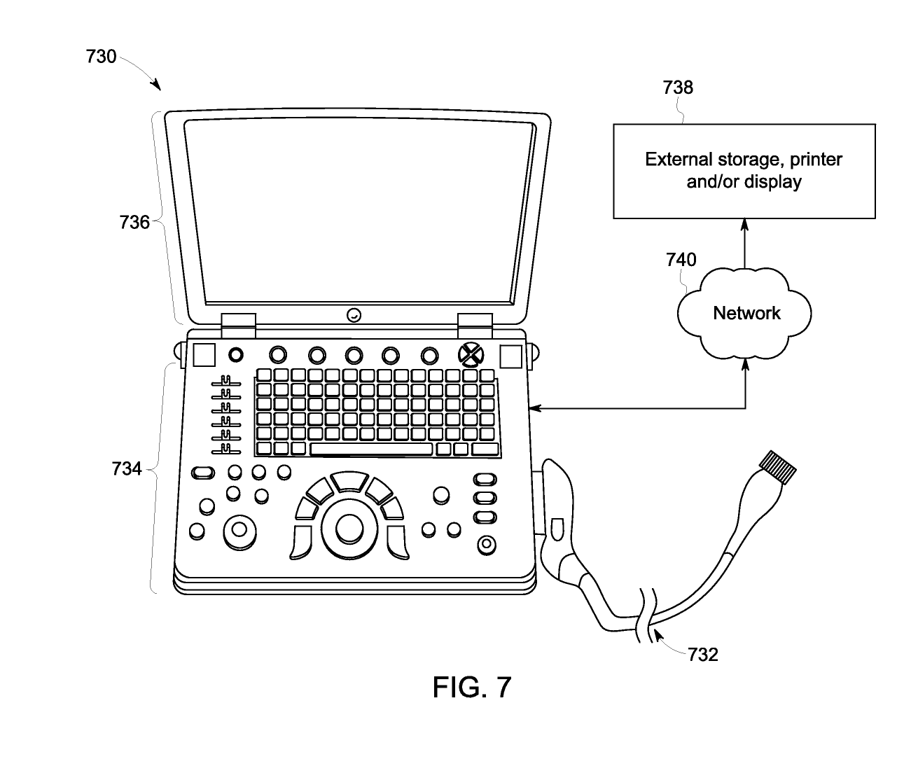

FIG. 2 illustrates a schematic block diagram of an ultrasound imaging system 200 according to one embodiment of the medical devices 102. The ultrasound imaging system 200 may be a unitary apparatus such that the elements and components of the system 200 may be carried or moved with each other. The ultrasound systems 730, 850, 900 shown in FIGS. 7, 8, and 9, respectively, illustrate examples of such systems. However, in other embodiments, at least one of the system components and elements described herein may be located remotely with respect to other components and elements. For example, one or more of the described operations and/or components may operate in a data server that has a distinct and remote location with respect to an ultrasound probe 226 and the user interface 242.

The ultrasound imaging system 200 includes an ultrasound probe 226 having a transmitter 222 and probe/SAP electronics 210. The ultrasound probe 226 may be configured to acquire ultrasound data or information from a region of interest (e.g., organ, blood vessel) of the patient. The ultrasound probe 226 is communicatively coupled to a controller circuit 236 via the transmitter 222. The transmitter 222 transmits a signal to a transmit beamformer 221 based on acquisition settings received by the user. The signal transmitted by the transmitter 222 in turn drives the transducer elements 224 within the transducer array 212. The transducer elements 224 emit pulsed ultrasonic signals into a patient (e.g., a body). A variety of a geometries and configurations may be used for the array 212. Further, the array 212 of transducer elements 224 may be provided as part of, for example, different types of ultrasound probes.

The acquisition settings may define an amplitude, pulse width, frequency, and/or the like of the ultrasonic pulses emitted by the transducer elements 224. The acquisition settings may be adjusted by the user by selecting a gain setting, power, time gain compensation (TGC), resolution, and/or the like from the user interface 242. Additionally or alternatively, the acquisition settings may be based and/or correspond to acquisition settings included within the PHI.

For example, in some embodiments, the controller circuit 236 may determine and/or detect the examination or scan to be performed based on information within the PHI. Based on the examination or scan to be performed, a table stored in the memory 240 is accessed by the controller circuit 236 to correlate the detected examination or scan, to one or more preset(s) configuration(s) of acquisition settings corresponding to the detected examination or scan.

The transducer elements 224, for example piezoelectric crystals, emit pulsed ultrasonic signals into a body (e.g., patient) or volume corresponding to the acquisition settings. The ultrasonic signals may include, for example, one or more reference pulses, one or more pushing pulses (e.g., shear-waves), and/or one or more tracking pulses. At least a portion of the pulsed ultrasonic signals back-scatter from a region of interest (ROI) (e.g., breast tissues, liver tissues, cardiac tissues, prostate tissues, and the like) to produce echoes. The echoes are delayed in time according to a depth, and are received by the transducer elements 224 within the transducer array 212. The ultrasonic signals may be used for imaging, for generating and/or tracking shear-waves, for measuring differences in compression displacement of the tissue (e.g., strain), and/or for therapy, among other uses. For example, the probe 226 may deliver low energy pulses during imaging and tracking, medium to high energy pulses to generate shear-waves, and high energy pulses during therapy.

The transducer array 212 may have a variety of array geometries and configurations for the transducer elements 224 which may be provided as part of, for example, different types of ultrasound probes 226. The probe/SAP electronics 210 may be used to control the switching of the transducer elements 224. The probe/SAP electronics 210 may also be used to group the transducer elements 224 into one or more sub-apertures.

The transducer elements 224 convert the received echo signals into electrical signals which may be received by a receiver 228. The electrical signals representing the received echoes are passed through a receive beamformer 230, which performs beamforming on the received echoes and outputs a radio frequency (RF) signal. The RF signal is then provided to an RF processor 232 that processes the RF signal. The RF processor 232 may generate different ultrasound image data types, e.g. B-mode, color Doppler (velocity/power/variance), tissue Doppler (velocity), and Doppler energy, for multiple scan planes or different scanning patterns. For example, the RF processor 232 may generate tissue Doppler data for multi-scan planes. The RF processor 232 gathers the information (e.g. I/Q, B-mode, color Doppler, tissue Doppler, and Doppler energy information) related to multiple data slices and stores the data information, which may include time stamp and orientation/rotation information, on the memory 234.

Alternatively, the RF processor 232 may include a complex demodulator (not shown) that demodulates the RF signal to form IQ data pairs representative of the echo signals. The RF or IQ signal data may then be provided directly to a memory 234 for storage (e.g., temporary storage). Optionally, the output of the beamformer 230 may be passed directly to the controller circuit 236.

The controller circuit 236 may be configured to process the acquired ultrasound data (e.g., RF signal data or IQ data pairs) and prepare frames of ultrasound image data for display on the display 238. The controller circuit 236 may include one or more processors. Optionally, the controller circuit 236 may include a central controller circuit (CPU), one or more microprocessors, a graphics controller circuit (GPU), or any other electronic component capable of processing inputted data according to specific logical instructions. Having the controller circuit 236 that includes a GPU may be advantageous for computation-intensive operations, such as volume-rendering. Additionally or alternatively, the controller circuit 236 may execute instructions stored on a tangible and non-transitory computer readable medium (e.g., the memory 240).

The controller circuit 236 is configured to perform one or more processing operations according to a plurality of selectable ultrasound modalities on the acquired ultrasound data, adjust or define the ultrasonic pulses emitted from the transducer elements 224, adjust one or more image display settings of components (e.g., ultrasound images, interface components) displayed on the display 238, and other operations as described herein. Acquired ultrasound data may be processed in real-time by the controller circuit 236 during a scanning or therapy session as the echo signals are received. Additionally or alternatively, the ultrasound data may be stored temporarily on the memory 234 during a scanning session and processed in less than real-time in a live or off-line operation.

The ultrasound imaging system 200 may include a memory 240 for storing processed frames of acquired ultrasound data that are not scheduled to be displayed immediately or to store post-processed images (e.g., shear-wave images, strain images), firmware or software corresponding to, for example, a graphical user interface, one or more default image display settings, and/or the like. The memory device 240 may be a tangible and non-transitory computer readable medium such as flash memory, RAM, ROM, EEPROM, and/or the like.

One or both of the memory 234 and 240 may store 3D ultrasound image data sets of the ultrasound data, where such 3D ultrasound image data sets are accessed to present 2D and 3D images. For example, a 3D ultrasound image data set may be mapped into the corresponding memory 234 or 240, as well as one or more reference planes. The processing of the ultrasound data, including the ultrasound image data sets, may be based in part on user inputs, for example, user selections received at the user interface 242.

The controller circuit 236 is operably coupled to a communication interface circuit 248. The communication interface circuit 248 may be controlled by the controller circuit 236 and be configured to establish and detect communication links (e.g., the one or more communication links 112) with the remote systems. For example, the communication interface circuit 248 may include physical layer (PHY) components such as a transceiver, one or more communication ports, a digital signal processor, one or more amplifiers, an antenna, and/or the like for communicatively coupling the ultrasound imaging system 200 to the remote systems. The communication interface circuit 248 may include one or more processors, a central controller circuit (CPU), one or more microprocessors, or any other electronic components capable of processing inputted data according to specific logical instructions.

The communication links established by the communication interface circuit 248 may conform to one or more communication protocols such as an Ethernet Standard, DICOM, USB, one or more wireless standards (e.g., 802.11, Bluetooth, Bluetooth Low Energy, ZigBee), and/or the like. The protocol firmware for the one or more communication protocols may be stored on the memory 240, which is accessible by the communication circuit 248 directly and/or via the controller circuit 236. Additionally or alternatively, the firmware may be stored within an internal memory of the communication interface circuit 248. The protocol firmware provide the communication protocol syntax for the communication interface circuit 248 to assemble data packets, establish one or more communication links, and/or partition data (e.g., PHI) received from the remote systems.

The communication link interface 248 is further configured to decrypt and/or encrypt data (e.g., PHI, updated PHI) along the one or more communication links based on the communication protocols used by the corresponding remote systems. For example, encryption may be based on pre-defined encryption algorithms stored in the memory 240. For example, the communication link interface 248 may use an Advanced Encryption Standard (AES) algorithm, an RSA algorithm standard (e.g., RSA-1024, RSA-2048), Secure Hash Algorithm (e.g., SHA-1, SHA-256, SHA-384, SHA-2), and/or the like on the PHI. In another example, a password based encryption may be used such as a PKCS series. Additionally or alternatively, the encryption may be based on a DICOM encryption standard, for example, as described in ISO standard 12052:2006 and NEMA standard PS3.

Additionally or alternatively, the communication interface circuit 248 may establish communication links with remote systems corresponding to peripheral devices communicably coupled via physical medium or wirelessly to the ultrasound imaging system 200. For example, the peripheral devices may include printers, USB devices (e.g., thumb drives, a computer mouse), scanners, barcode readers, and/or the like. One or more of the communication links with the peripheral devices established by the communication interface circuit 248 may be included with a user interface 242.

The controller circuit 236 is operably coupled to a display 238 and a user interface 242. The display 238 may include one or more liquid crystal displays (e.g., light emitting diode (LED) backlight), organic light emitting diode (OLED) displays, plasma displays, CRT displays, and/or the like. The display 238 may display patient information, a PHI workflow, ultrasound images and/or videos, components of a display interface, one or more 2D, 3D, or 4D ultrasound image data sets from ultrasound data stored on the memory 234 or 240 or currently being acquired, measurements, diagnosis, treatment information, and/or the like received by the display 238 from the controller circuit 236.

The user interface 242 may include hardware, firmware, software, or a combination thereof that enables an individual (e.g., an operator) to directly or indirectly control operation of the ultrasound system 200 and the various components thereof. The user interface 242 controls operations of the controller circuit 236 and is configured to receive inputs from the user. For example, the user interface 242 may include a keyboard, a mouse, a touchpad, one or more physical buttons, and/or the like. Optionally, the display 238 may be a touch screen display, which includes at least a portion of the user interface 242 shown as a graphical user interface (GUI). The touch screen display can detect a presence of a touch from the operator on the display 238 and can also identify a location of the touch in the display 238. For example, the user may select one or more user selectable elements shown on the display by touching or making contact with the display 238. The touch may be applied by, for example, at least one of an individual's hand, glove, stylus, or the like.

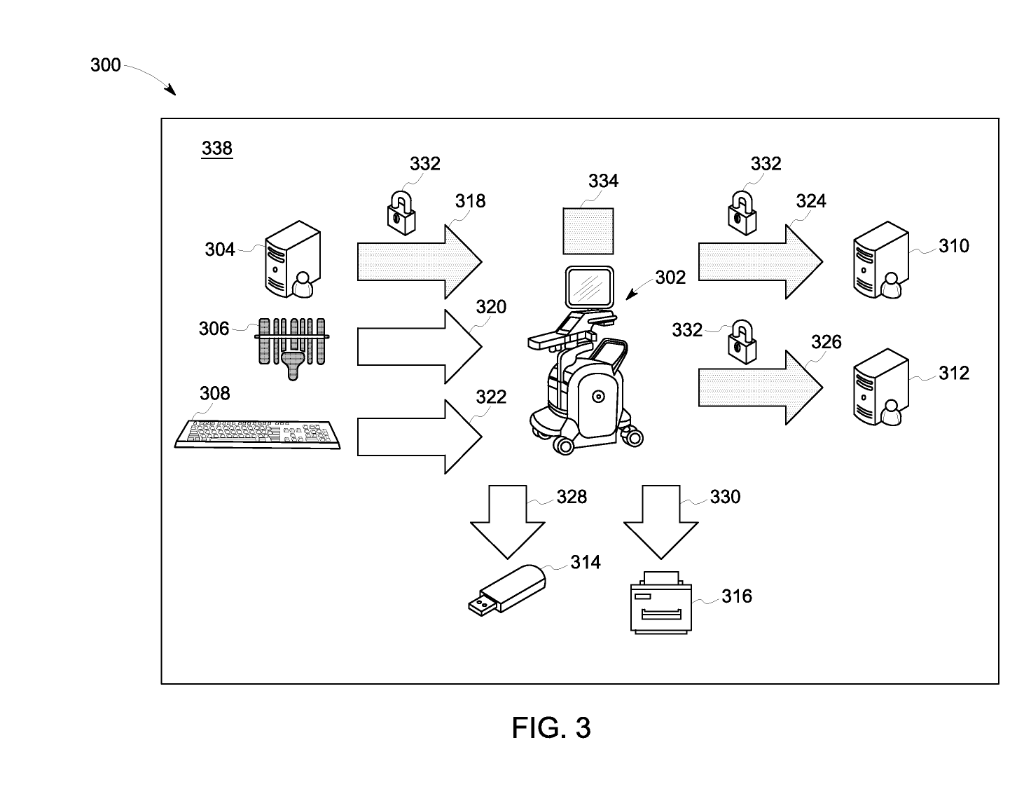

In various embodiments the user interface 242 (e.g., GUI) and the display 238 may communicates information to the operator by displaying the information to the operator. For example, the display 238 may present information to the operator during the imaging session. The information presented may include ultrasound images, graphical elements, user-selectable elements, and other information (e.g., administrative information, personal information of the patient, and the like). In connection with FIG. 3, the display 238 can present information corresponding to a PHI workflow 338 of the ultrasound imaging system 200.

FIG. 3 illustrates a screenshot 300 of the PHI workflow 338 shown on the display 238 in accordance with an embodiment. The PHI workflow 338 is shown as an object object-oriented visualization. For example, the PHI workflow 338 includes graphical icons 304-316 and connection graphics 318-330, which visually illustrates movement and/or transmission of the PHI with respect to a medical device 102 (e.g., the ultrasound imaging system 200).

The PHI workflow 338 includes remote systems, peripheral devices, and the ultrasound imaging system 200 displayed as corresponding graphical icons 304-316. For example, the graphical icon 302 may represent the ultrasound imaging system 200 (e.g., one of the medical devices 102), the graphical icon 304 may represent the patient reference system 104 (FIG. 1), the graphical icon 310 may represent the billing system 108, and the graphical icon 312 may represent the PACS 110. The graphical icons 306-308 and 314-316 may correspond to remote systems that are peripherals communicatively coupled to the ultrasound imaging system 200. For example, the graphical icon 306 may represent a barcode reader, the graphical icon 308 may represent a keyboard (e.g., such as part of the user interface 242), the graphical icon 314 may represent an external memory storage (e.g., USB thumbdrive), and the graphical icon 316 may represent a printer. Optionally, the PHI workflow 338 may include a graphical icon 334 to indicate whether PHI stored on the memory 240 of the ultrasound imaging system 200 is encrypted.

The graphical icons 304-316 are connected to the ultrasound imaging system 200 via communication links, which are shown as connection graphics 318-330. The connection graphics 318-330 are illustrated as arrows to illustrate a flow and/or direction of the PHI between the ultrasound imaging system 200 and the remote system. For example, the connection graphic 318 shows a direction of the arrow towards the ultrasound imaging system 200, represented as the graphical icon 302, to illustrates that the ultrasound imaging system 200 may receive the PHI from the patient reference system 104 (represented as the graphical icon 304). Similarly, the connection graphics 320-322 shows a direction of the arrow towards the graphical icon 302, to illustrate that the ultrasound imaging system 200 may receive the PHI from the barcode reader (represented as the graphical icon 306) when scanning a barcode label and the keyboard (represented as the graphical icon 308).

In another example, the direction of the arrow of the connection graphics 324-330 shows the ultrasound imaging system 200 can transmit the updated PHI to the billing system 108 (represented as the graphical icon 310), the PACS 110 (represented as the graphical icon 312), an external memory storage (represented as the graphical icon 314), and/or the printer (represented as the graphical icon 316).

It should be noted in other embodiments a position of the graphical icons 304-316 with respect to the graphical icon 302 may be used to illustrate flow and/or direction of the PHI. Additionally or alternatively, a visual feature, shape, and/or size of the graphical icons 304-316 with respect to each other may be used to illustrate a flow and/or direction of the PHI with respect to the medical device.

The connection graphics 318-330 may have visual features corresponding to an encryption level of the communication link represented by the connection graphic 318-330. The visual feature may be a color, an animation (e.g., scrolling, flashing), a graphical icon (e.g., the graphical icon 332), and/or the like. The encryption level may correspond to when the PHI and/or the transmission of data along the communication link is encrypted. For example, a color of the connection graphics 318, 324, and 326 indicate that the associated communication links are encrypted. In another example, a color of the connection graphics 320-322, and 328-330 indicate that the associated communication links are not encrypted.

The encryption of the communication link may correspond to an Advanced Encryption Standard (AES) algorithm, an RSA algorithm standard (e.g., RSA-1024, RSA-2048), Secure Hash Algorithm (e.g., SHA-1, SHA-256, SHA-384, SHA-2), and/or the like on the PHI. In another example, a password based encryption may be used such as a PKCS series. Additionally or alternatively, the encryption may be based on a DICOM encryption standard, for example, as described in ISO standard 12052:2006 and NEMA standard PS3. It should be noted that in various embodiments, the encryption level may correspond to a type and/or level of encryption used along the communication link. For example, one of the encryption levels may correspond to a key size of the encryption used, the standard of encryption used, and/or the like.

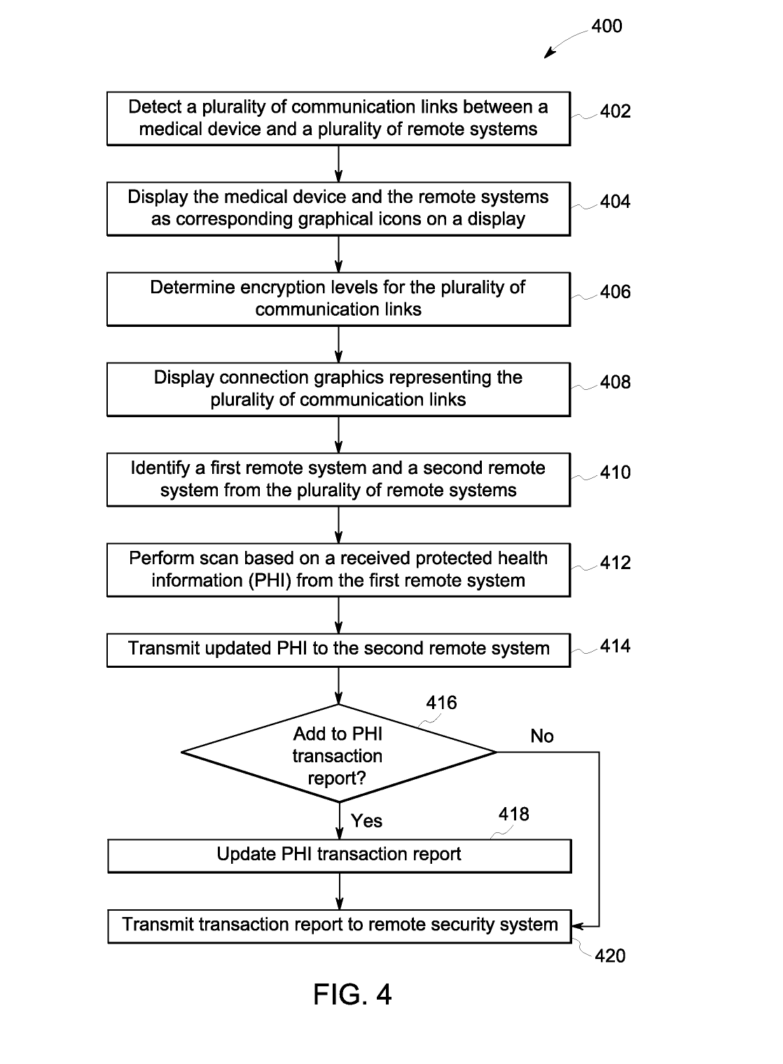

In connection with FIG. 4, the PHI workflow 338 may be configured by the controller circuit 236 and/or based on a predetermined PHI workflow stored on the memory 240. Optionally, the PHI workflow 338 may be adjusted and/or managed by the user using the user interface 242.

FIG. 4 illustrates a flowchart of a method 400 for managing PHI, in accordance with various embodiments described herein. The method 400, for example, may employ structures or aspects of various embodiments (e.g., systems and/or methods) discussed herein. In various embodiments, certain steps (or operations) may be omitted or added, certain steps may be combined, certain steps may be performed simultaneously, certain steps may be performed concurrently, certain steps may be split into multiple steps, certain steps may be performed in a different order, or certain steps or series of steps may be re-performed in an iterative fashion. In various embodiments, portions, aspects, and/or variations of the method 400 may be used as one or more algorithms to direct hardware to perform one or more operations described herein. It should be noted, other methods may be used, in accordance with embodiments herein.

One or more methods may (i) detect a plurality of communication links between a medical device and a plurality of remote systems; (ii) display the medical device and the remote systems as corresponding graphical icons on a display; (iii) determine encryption levels for the plurality of communication links; and (iv) display connection graphics representing the plurality of communication links.

Beginning at 402, the controller circuit 236 may detect a plurality of communication links between a medical device (e.g., the ultrasound imaging system 200, the medical device 102) and a plurality of remote systems (e.g., a patient reference system 104, a billing system 108, a picture archive communication system 110, peripheral devices). The detected communication links may correspond to remote systems that the communication interface circuit of the medical device can transmit and/or receive data, such as PHI.

For example, the controller circuit 236 may instruct the communication interface circuit 248 to transmit an advertisement packet (e.g., connection status request, connection request) from communication ports of the ultrasound imaging system 200, at least a portion of which are communicatively coupled to one or more of the remote devices. When the communication interface circuit 248 receives a response from the remote devices, the communication interface circuit 248 may send a detection signal corresponding to detection of communication links with the responding remote devices.

In another example, the plurality of communication links may be predetermined based on a default communication configuration stored on the memory 240. The default communication configuration may include a listing of remote devices that are communicatively coupled to the ultrasound imaging system 200.

At 404, the medical device and the remote system are displayed as corresponding graphical icons on a display. For example, based on the responding remote devices at 402, the controller circuit 236 may send a display signal to the display 238. The display signal may be a video interface (e.g., Video Graphics Array, DisplayPort, High Definition Multimedia Interface, Digital Visual Interface, MHL, SDI, and/or the like) which is used by the display 238. The display signal may correspond to a series of pixel configurations from the controller circuit 236 forming the PHI workflow 338 on the display 238. For example, the controller circuit 236 may retrieve pixel information corresponding to the graphical icons 304-316 stored in the memory 240. The controller circuit 236 may include the pixel information of the graphical icons 304-316 within the display signal, which will be displayed by the display 238 when the display signal is received.

At 406, the controller circuit 236 may determine encryption levels for the plurality of communication links. The encryption level may correspond to a presence and/or use of an encryption of the PHI or updated PHI when transmitted along the communication link. The controller circuit 236 may determine which of the communication links are encrypted based on the communication protocol (e.g., DICOM) used for the communication link and/or if pre-determined encryption algorithms are being used by the communication protocol interface 238 for the communication link.

For example, the controller circuit 236 may determine that the communication link with the patient reference system 104 represented by the graphical icon 304 is encrypted, since the communication link uses a DICOM protocol. In another example, the controller circuit 236 may determine that the communication link with the billing system 108 represented by the graphical icon 310 is encrypted, since the communication interface circuit 238 uses an AES algorithm to encrypt the data before transmitting to the billing system 108. In another example, the controller circuit 236 may determine that the communication link with the external memory storage represented by the graphical icon 314 is not encrypted, since the communication link uses a USB protocol and/or does not use a pre-determined encryption algorithm.

At 408, the controller circuit 236 may display connection graphics 318-330 representing the plurality of communication links. As shown in FIG. 3, the connection graphics 318-330 may each be positioned between the medical device (e.g., represented by the graphical icon 302) and one of the remote system. The connection graphics 318-330 each have a visual feature (e.g., color, animation, graphical icon) corresponding to an encryption level of the communication link between the medical device and the one of the remote system represented by the connection graphic 318-330. For example, a color of the connection graphics 318, 324, and 326 indicate that the associated communication links between the ultrasound system 200 (represented by the graphical icon 302) and the patient reference system 104 (represented by the graphical icon 304), the billing system 108 (represented by the graphical icon 310), and the PACS 110 (represented by the graphical icon 312), respectively, are encrypted.

At 410, the controller circuit 236 may identify a first remote system and a second remote system from the plurality of remote system. The medical device may receive the PHI from the first remote system, and transmit the updated PHI to the second remote system. In various embodiments, the controller circuit 236 may identify the first and second remote system based on a user selection via the user interface 242. For example, in connection with FIG. 3, the user may select one of the graphical icons 304-308 and/or connection graphics 318-322 shown on the display 238 via the user interface 242 (e.g., the touchscreen, the trackpad, the keyboard, the mouse) that correspond to the remote systems that the medical device receives the PHI (e.g., based on the arrow direction of the graphical icons 304-308) as the first remote system. Additionally, the user may select one or more of the graphical icons 310-316 and/or connection graphics 324-330 via the user interface 242 that correspond to the remote system that the medical device transmits the updated PHI as the second remote system.

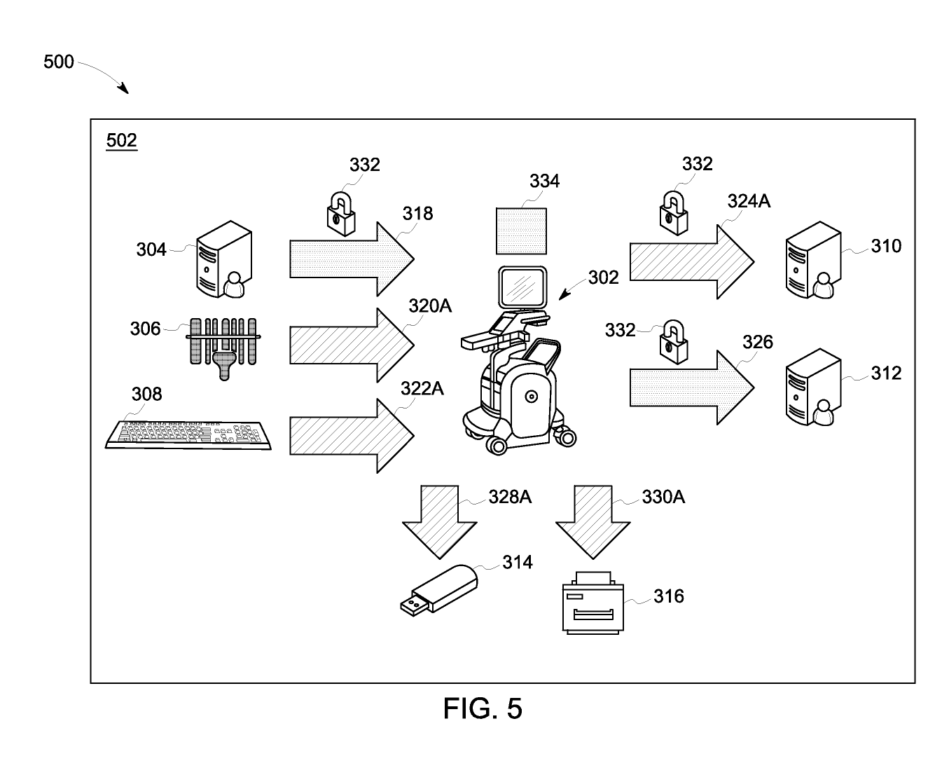

Additionally or alternatively, in connection with FIG. 5, the controller circuit 236 may identify the first remote system and the second remote system based on a predetermined PHI workflow.

The predetermined PHI workflow may correspond to a rule set based on the user of the medical device. For example, medical students may have a different predetermined PHI workflow than attending physicians. In another example, users within different medical departments may receive and/or transmit the PHI and updated PHI, respectively, to different remote systems. Optionally, the predetermined PHI workflow may correspond to a security policy on allowable remote systems to receive PHI and/or transmit updated PHI from the medical device.

In various other embodiments, the predetermined PHI workflow may be uploaded to the medical devices 102, for example, from the monitoring system 106, from a boot disk, and/or the like. Additionally or alternatively, the predetermined PHI workflow may be defined by the user and/or a medical administrator.

For example, the medical administrator may enable a configuration mode of the ultrasound imaging system 200. During the configuration mode, the medical administrator may define and/or configure a default predetermined PHI workflow for the ultrasound imaging system 200 for all users and/or select one or more users corresponding to the newly defined predetermine PHI workflow. To define the predetermined PHI work flow, the medical administrator may select one or more of the graphical icons 304-308 and/or connection graphics 318-322 shown on the display 238 via the user interface 242 (e.g., the touchscreen, the trackpad, the keyboard, the mouse) that correspond to the remote systems that the medical device receives the PHI (e.g., based on the arrow direction of the graphical icons 304-308) as the first remote system. Additionally, the medical administrator may select one or more of the graphical icons 310-316 and/or connection graphics 324-330 via the user interface 242 that correspond to the remote system that the medical device transmits the updated PHI as the second remote system. Optionally, the medical administrator may create a priority list for the different remote systems to set a rule set for the predetermined PHI workflow. When the predetermined PHI workflow is defined, the medical administrator may exit the configuration mode using the user interface 242, and save the predetermined PHI workflow in the memory 240 and/or save on a remote system (e.g., the monitoring system 106).

The rule set of the predetermined PHI workflow may be used by the controller circuit 236 to identify which remote systems having a communication link with the medical device correspond to the first remote system and the second remote system. For example, users can log into the medical device using a username via the user interface 242. The controller circuit 236 may compare the username with a login configuration database stored in the memory 240. The login configuration database may be a collection of candidate predetermined PHI workflows with corresponding usernames. The controller circuit 236 may select one of the candidate predetermined PHI workflows that match the username of the user and adjust the PHI workflow 338 and/or generate a PHI workflow based on the predetermined PHI workflow for the display 238.

FIG. 5 illustrates a screenshot 500 of a PHI workflow 502 shown on the display 238 based on a predetermined PHI workflow in accordance with an embodiment. For example, the username entered by the user corresponds to a predetermined PHI workflow that restricts the medical device from receiving PHI and/or transmitting the updated PHI along an unencrypted communication link, which corresponds to the communication links represented by the connection graphics 320a-322a and 328a-330a. Additionally or alternatively, the predetermined PHI workflow may indicate which communication links are preferred and/or do not conform to the security policy of the medical network 100. Optionally, the connection graphics 320a-324a and 328a-330a may include a visual feature, such as a color pattern as illustrated in FIG. 5, corresponding to the disabled and/or non-conforming communication links between the remote systems and the medical device. Based on the predetermined workflow, specifically the remaining enabled and/or conforming communication links, the controller circuit 236 may determine that the first remote system corresponds to the graphical icon 304 and the second remote system corresponds to the graphical icon 312.

Additionally or alternatively, the user may override the predetermined PHI workflow. For example, the user may select one or more of the graphical icons 306-308, 310, 314-316 and/or connection graphics 320a-324a, 328a-330a shown on the display 238 via the user interface 242 (e.g., the touchscreen, the trackpad, the keyboard, the mouse) representing a remote system having a disabled and/or non-conforming communication link as the first and/or second remote device.

At 412, the controller circuit 236 performs a scan based on the received PHI from the first remote system. For example, the first remote system (e.g., the patient reference system 104) may transmit the PHI to the ultrasound imaging system 200. The PHI may include the type of examination and/or scan to be performed by the ultrasound imaging system 200. The controller circuit 236 may receive the PHI and display portions of the PHI on the display (e.g., name of the patient, scan to be performed) and/or automatically adjust the acquisition settings of the ultrasound probe 226 based on the scan information included within the PHI to prepare the ultrasound imaging system 200 for the scan. Optionally, the user may adjust the acquisition settings manually via the user interface 242. In various embodiments, when the acquisition settings are configured for the scan described in the PHI, the ultrasound probe 226 may emit the pulses ultrasound signals into the patient from the transducer elements 224 to initiate the scan described in the PHI.

At 414, the controller circuit 236 transmits the updated PHI to the second remote system. The updated PHI may include the medical a data acquired by the controller circuit 236 during the scan performed at 412. For example, the controller circuit 236 may acquire one or more ultrasound images from the scan performed at 412. The controller circuit 236 may add and/or burn portions of the PHI into the one or more ultrasound images to form the updated PHI. Additionally or alternatively, the controller circuit 236 may include timing and location information on when the scan was performed, which medical device 102 performed the scan, the user of the scanning medical device 102, and/or the like to the PHI received from the first remote system to form the updated PHI.

At 416, the controller circuit 236 determines whether to add the updated PHI transmission to a PHI transaction report. The PHI transaction report may be stored on the memory 240. The PHI transaction report may be a collection of transmission information of the updated PHI from the ultrasound imaging system 200 to a second remote system. For example, the transmission information may include a portion of the PHI, such as a patient name and/or scanning information corresponding to the updated PHI, a time stamp of the transmission, encryption information, and the intended remote system (e.g., the second remote system) receiving the updated PHI. In various embodiments the controller circuit 236 may add each updated PHI transmission to the PHI transaction report.

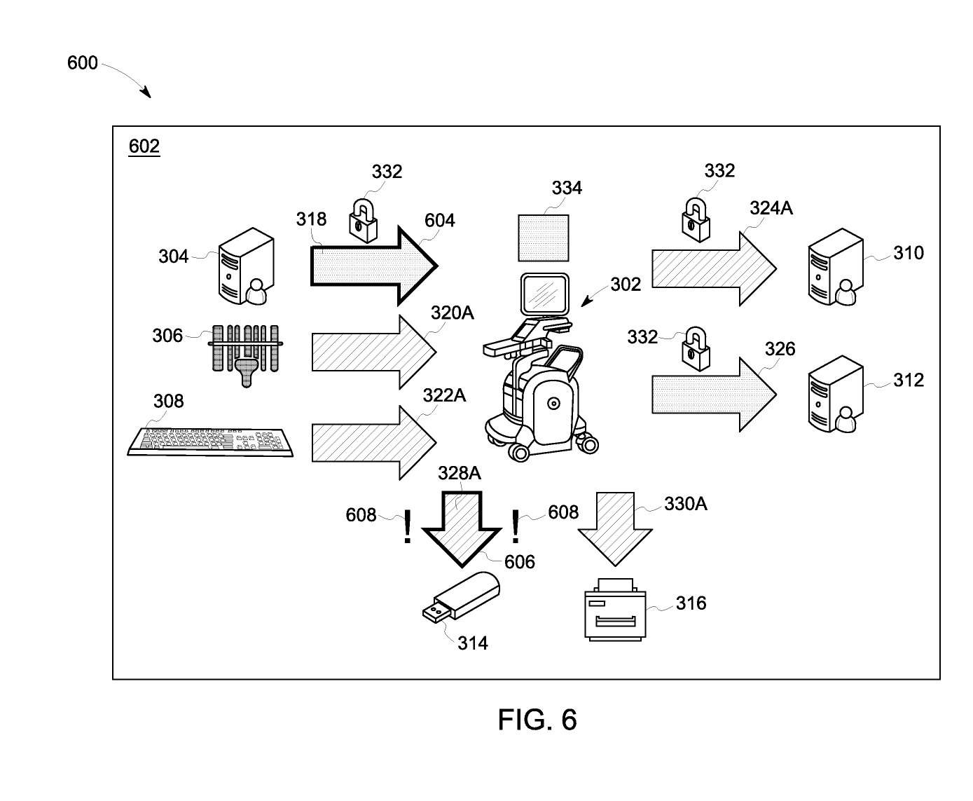

Additionally or alternatively, in connection with FIG. 6, the controller circuit 236 may add updated PHI transmissions to the PHI transaction report when the transmission was against a PHI predetermined workflow and/or security policy of the ultrasound imaging system 200.

FIG. 6 illustrates a screenshot 600 of a PHI workflow 602 shown on the display 238 based on the predetermined PHI workflow used in FIG. 5. The first remote system, corresponding to the graphical icon 304, and the second remote system, corresponding to the graphical icon 314, are shown on the PHI workflow 602 having connection graphics 318 and 328a with visual features 604 and 606 (e.g. highlighted outlines), respectively. For example, the controller circuit 236 may have identified the first remote system corresponding to the graphical icon 304 based on the predetermined PHI workflow as described in connection with FIG. 5. The second remote system may have been selected by the user using the user interface 242, contrary to the predetermined PHI workflow as described above. Optionally, the controller circuit 236 may have visual alerts 608 positioned around and/or proximate to the connection graphic 328a to indicate that the selected second remote device is against and/or contradicted by the predetermined PHI workflow.

The controller circuit 236 may determine that since the second remote system is contrary to the predetermined PHI workflow, a transmission of the updated PHI to the second remote system will be added to the PHI transaction report.

If the updated PHI transmission is determined to be added to the PHI transaction report, then at 418, the controller circuit 236 updates the PHI transaction report. For example, when the updated PHI is transmitted from the ultrasound imaging system 200, the controller circuit 236 may add identification information of the second remote system (e.g., port address, network name, network address) and corresponding patient information (e.g., name of the patient) from the updated PHI to the PHI transaction report.

At 420, the controller circuit 236 transmits the PHI transaction report to a remote security system. The controller circuit 236 may transmit the PHI transaction report stored on the memory 240 periodically (e.g., at predetermined day and/or hour) to the monitoring system 106 and/or automatically when the PHI transaction report is updated at 418. For example, the ultrasound imaging system 200 may be communicatively coupled to the monitoring system 106 along a communication link established by the communication interface circuit 248.

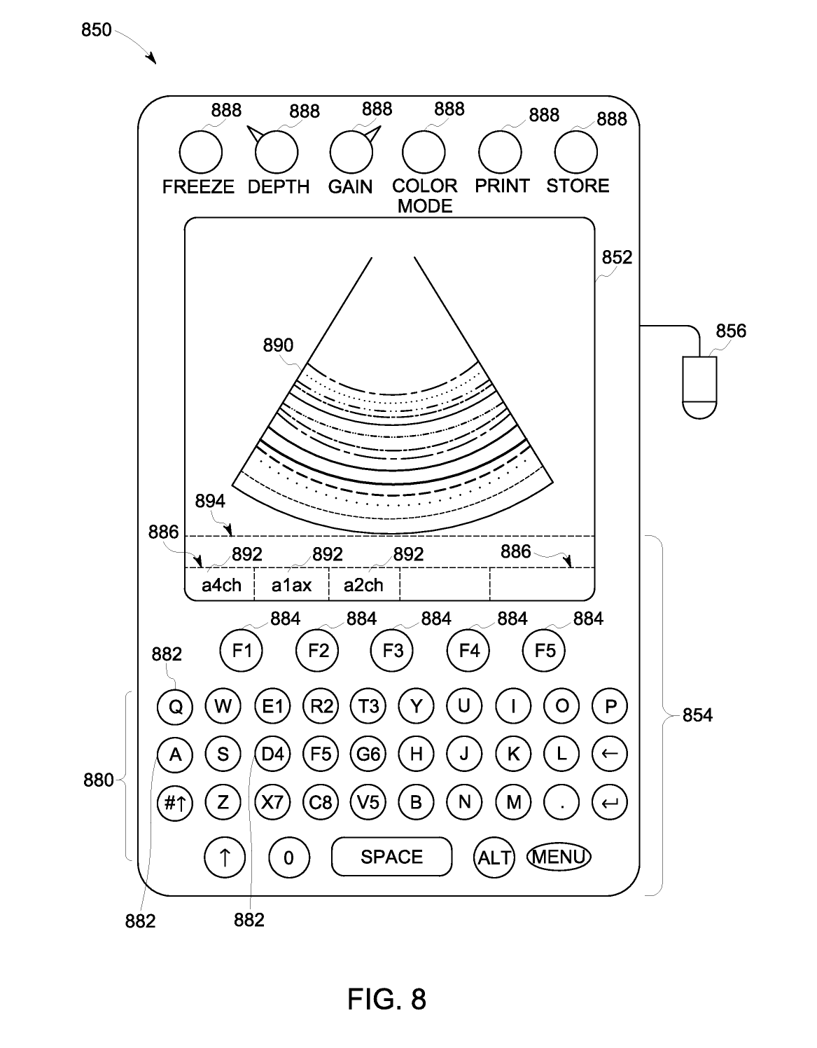

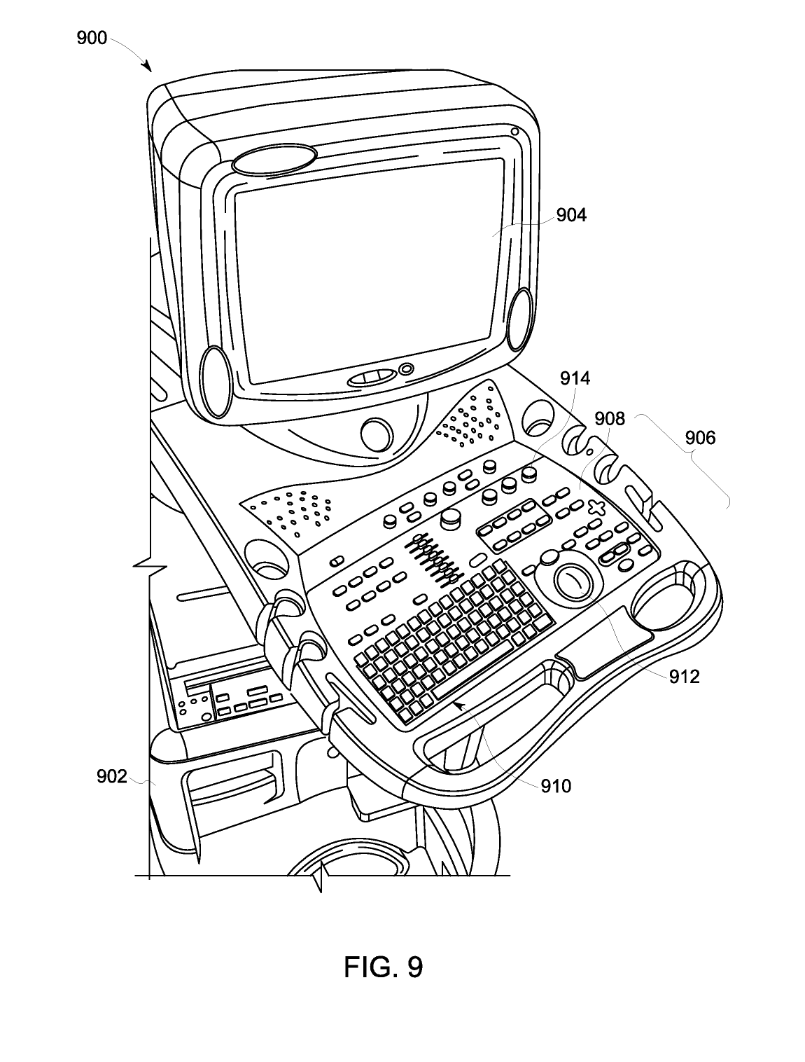

The ultrasound system 200 of FIG. 2 may be embodied in a small-sized system, such as laptop computer or pocket-sized system as well as in a larger console-type system. FIGS. 7 and 8 illustrate small-sized systems, while FIG. 9 illustrates a larger system.

FIG. 7 illustrates a 3D-capable miniaturized ultrasound system 730 having a probe 732 that may be configured to acquire 3D ultrasonic data or multi-plane ultrasonic data. For example, the probe 732 may have a 2D array of elements as discussed previously with respect to the probe. A user interface 734 (that may also include an integrated display 736) is provided to receive commands from an operator. As used herein, “miniaturized” means that the ultrasound system 730 is a handheld or hand-carried device or is configured to be carried in a person's hand, pocket, briefcase-sized case, or backpack. For example, the ultrasound system 730 may be a hand-carried device having a size of a typical laptop computer. The ultrasound system 730 is easily portable by the operator. The integrated display 736 (e.g., an internal display) is configured to display, for example, one or more medical images.

The ultrasonic data may be sent to an external device 738 via a wired or wireless network 740 (or direct connection, for example, via a serial or parallel cable or USB port). In some embodiments, the external device 738 may be a computer or a workstation having a display. Alternatively, the external device 738 may be a separate external display or a printer capable of receiving image data from the hand carried ultrasound system 730 and of displaying or printing images that may have greater resolution than the integrated display 736.

FIG. 8 illustrates a hand carried or pocket-sized ultrasound imaging system 850 wherein the display 852 and user interface 854 form a single unit. By way of example, the pocket-sized ultrasound imaging system 850 may be a pocket-sized or hand-sized ultrasound system approximately 2 inches wide, approximately 4 inches in length, and approximately 0.5 inches in depth and weighs less than 3 ounces. The pocket-sized ultrasound imaging system 850 generally includes the display 852, user interface 854, which may or may not include a keyboard-type interface and an input/output (I/O) port for connection to a scanning device, for example, an ultrasound probe 856. The display 852 may be, for example, a 320×320 pixel color LCD display (on which a medical image 890 may be displayed). A typewriter-like keyboard 880 of buttons 882 may optionally be included in the user interface 854.

Multi-function controls 884 may each be assigned functions in accordance with the mode of system operation (e.g., displaying different views). Therefore, each of the multi-function controls 884 may be configured to provide a plurality of different actions. One or more interface components, such as label display areas 886 associated with the multi-function controls 884 may be included as necessary on the display 852. The system 850 may also have additional keys and/or controls 888 for special purpose functions, which may include, but are not limited to “freeze,” “depth control,” “gain control,” “color-mode,” “print,” and “store.”

One or more of the label display areas 886 may include labels 892 to indicate the view being displayed or allow a user to select a different view of the imaged object to display. The selection of different views also may be provided through the associated multi-function control 884. The display 852 may also have one or more interface components corresponding to a textual display area 894 for displaying information relating to the displayed image view (e.g., a label associated with the displayed image).

It should be noted that the various embodiments may be implemented in connection with miniaturized or small-sized ultrasound systems having different dimensions, weights, and power consumption. For example, the pocket-sized ultrasound imaging system 850 and the miniaturized ultrasound system 830 may provide the same scanning and processing functionality as the system 100.

FIG. 9 illustrates an ultrasound imaging system 900 provided on a movable base 902. The portable ultrasound imaging system 900 may also be referred to as a cart-based system. A display 904 and user interface 906 are provided and it should be understood that the display 904 may be separate or separable from the user interface 906. The user interface 906 may optionally be a touchscreen, allowing the operator to select options by touching displayed graphics, icons, and the like.

The user interface 906 also includes control buttons 908 that may be used to control the portable ultrasound imaging system 900 as desired or needed, and/or as typically provided. The user interface 906 provides multiple interface options that the user may physically manipulate to interact with ultrasound data and other data that may be displayed, as well as to input information and set and change scanning parameters and viewing angles, etc. For example, a keyboard 910, trackball 912 and/or multi-function controls 914 may be provided.

It should be noted that although the various embodiments may be described in connection with an ultrasound system, the methods and systems are not limited to ultrasound imaging or a particular configuration thereof. The various embodiments may be implemented in connection with different types of diagnostic medical imaging systems, including, for example, x-ray imaging systems, magnetic resonance imaging (MRI) systems, computed-tomography (CT) imaging systems, positron emission tomography (PET) imaging systems, or combined imaging systems, among others.

It should be noted that the various embodiments may be implemented in hardware, software or a combination thereof. The various embodiments and/or components, for example, the modules, or components and controllers therein, also may be implemented as part of one or more computers or processors. The computer or processor may include a computing device, an input device, a display unit and an interface, for example, for accessing the Internet. The computer or processor may include a microprocessor. The microprocessor may be connected to a communication bus. The computer or processor may also include a memory. The memory may include Random Access Memory (RAM) and Read Only Memory (ROM). The computer or processor further may include a storage device, which may be a hard disk drive or a removable storage drive such as a solid-state drive, optical disk drive, and the like. The storage device may also be other similar means for loading computer programs or other instructions into the computer or processor.

As used herein, the term “computer,” “subsystem” or “module” may include any processor-based or microprocessor-based system including systems using microcontrollers, reduced instruction set computers (RISC), ASICs, logic circuits, and any other circuit or processor capable of executing the functions described herein. The above examples are exemplary only, and are thus not intended to limit in any way the definition and/or meaning of the term “computer”.

The computer or processor executes a set of instructions that are stored in one or more storage elements, in order to process input data. The storage elements may also store data or other information as desired or needed. The storage element may be in the form of an information source or a physical memory element within a processing machine.

The set of instructions may include various commands that instruct the computer or processor as a processing machine to perform specific operations such as the methods and processes of the various embodiments. The set of instructions may be in the form of a software program. The software may be in various forms such as system software or application software and which may be embodied as a tangible and non-transitory computer readable medium. Further, the software may be in the form of a collection of separate programs or modules, a program module within a larger program or a portion of a program module. The software also may include modular programming in the form of object-oriented programming. The processing of input data by the processing machine may be in response to operator commands, or in response to results of previous processing, or in response to a request made by another processing machine.

As used herein, a structure, limitation, or element that is “configured to” perform a task or operation is particularly structurally formed, constructed, or adapted in a manner corresponding to the task or operation. For purposes of clarity and the avoidance of doubt, an object that is merely capable of being modified to perform the task or operation is not “configured to” perform the task or operation as used herein. Instead, the use of “configured to” as used herein denotes structural adaptations or characteristics, and denotes structural requirements of any structure, limitation, or element that is described as being “configured to” perform the task or operation. For example, a controller circuit, processor, or computer that is “configured to” perform a task or operation may be understood as being particularly structured to perform the task or operation (e.g., having one or more programs or instructions stored thereon or used in conjunction therewith tailored or intended to perform the task or operation, and/or having an arrangement of processing circuitry tailored or intended to perform the task or operation). For the purposes of clarity and the avoidance of doubt, a general purpose computer (which may become “configured to” perform the task or operation if appropriately programmed) is not “configured to” perform a task or operation unless or until specifically programmed or structurally modified to perform the task or operation.

As used herein, the terms “software” and “firmware” are interchangeable, and include any computer program stored in memory for execution by a computer, including RAM memory, ROM memory, EPROM memory, EEPROM memory, and non-volatile RAM (NVRAM) memory. The above memory types are exemplary only, and are thus not limiting as to the types of memory usable for storage of a computer program.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the various embodiments without departing from their scope. While the dimensions and types of materials described herein are intended to define the parameters of the various embodiments, they are by no means limiting and are merely exemplary. Many other embodiments will be apparent to those of skill in the art upon reviewing the above description. The scope of the various embodiments should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means-plus-function format and are not intended to be interpreted based on 35 U.S.C. §112(f) unless and until such claim limitations expressly use the phrase “means for” followed by a statement of function void of further structure.

This written description uses examples to disclose the various embodiments, including the best mode, and also to enable any person skilled in the art to practice the various embodiments, including making and using any devices or systems and performing any incorporated methods. The patentable scope of the various embodiments is defined by the claims, and may include other examples that occur to those skilled in the art. Such other examples are intended to be within the scope of the claims if the examples have structural elements that do not differ from the literal language of the claims, or the examples include equivalent structural elements with insubstantial differences from the literal language of the claims.

What is claimed is:

1. A method for managing protected health information on a medical display, comprising:
detecting a plurality of communication links between a medical device and a plurality of remote systems;
displaying the medical device and the remote systems as corresponding graphical icons on a display;
determining encryption levels for the plurality of communication links;
displaying connection graphics representing the plurality of communication links, each connection graphic is positioned between the medical device and one of the remote systems having a visual feature corresponding to an encryption level of a communication link between the medical device and the one of the remote systems.

2. The method of claim 1, further comprising receiving a predetermined protected health information (PHI) workflow identifying a first remote system and a second remote system from the plurality of remote systems, wherein the medical device receives PHI from the first remote system and transmits an updated PHI to the second remote system.

3. The method of claim 2, further comprising adding identification information of the second remote system and corresponding patient information from the updated PHI to a PHI transaction report when the medical device transmits the updated PHI.

4. The method of claim 3, further comprising transmitting the PHI transaction report along to a monitoring system.

5. The method of claim 2, wherein the updated PHI includes at least one medical image acquired by the medical device.

6. The method of claim 2, wherein the second remote system is a picture archiving and communication system (PACS).

7. The method of claim 1, wherein the PHI includes at least one of a name of a patient, examination information, a patient characteristic, a Digital Imaging and Communications in Medicine (DICOM) worklist, or a patient medical history.

8. The method of claim 1, wherein the encryption levels corresponds to a Digital Imaging and Communications in Medicine (DICOM) encryption standard.

9. The method of claim 1, wherein the visual features correspond to a color, a graphical icon, or a visual animation.

10. The method of claim 1, wherein the medical device is an ultrasound system and the PHI includes one or more probe acquisition parameters.

11. An ultrasound imaging system comprising:
a display;
a communication interface circuit configured to establish a first communication link for receiving protected health information (PHI) from a first remote system and a second communication link for transmitting updated PHI to a second remote system;
a memory configured to store programmed instructions;
one or more processors to execute the programmed instructions by performing the following operations:
displaying on the display graphical icons corresponding to an ultrasound imaging system, the first remote system, and the second remote system;
determining encryption levels of the first communication link and the second communication link; and
displaying a first connection graphic representing the first communication link and a second connection graphic representing the second communication link on the display, the first connection graphic including at least one first visual feature corresponding to a first encryption level of the first communication link, and the second connection graphic including at least one second visual feature corresponding to a second encryption level of the second communication link.

12. The ultrasound imaging system of claim 11, the one or more processors further adding identification information of the second remote system and corresponding patient information from the updated PHI to a PHI transaction report stored in the memory when the medical device transmits the updated PHI.

13. The ultrasound imaging system of claim 11, wherein the communication interface circuit is configured to establish a third communication link to a monitoring system; and
the one or more processors further transmit the PHI transaction report along the third communication link to the monitoring system.

14. The ultrasound imaging system of claim 11, further comprising:
a user interface,
the one or more processors further receiving a predetermined PHI workflow via the user interface and selecting the first remote system and the second remote system from a group of remote system based on the predetermined PHI workflow configuration.

15. The ultrasound imaging system of claim 11, wherein the PHI includes at least one of a name of a patient, examination information, a patient characteristic, a Digital Imaging and Communications in Medicine (DICOM) worklist, or a patient medical history.

16. The ultrasound imaging system of claim 11, wherein the at least one of the first encryption level or the second encryption level corresponds to the PHI being encrypted by a Digital Imaging and Communications in Medicine (DICOM) standard.

17. The ultrasound imaging system of claim 11, further comprising an ultrasound probe configured to acquire ultrasound data,
the one or more processors further generating one or more medical images based on the acquired ultrasound data, wherein the updated PHI includes the one or more medical images.

18. A tangible and non-transitory computer readable medium comprising one or more programmed instructions configured to direct one or more processors to:
detect a plurality of communication links between a medical device and a plurality of remote systems;
display the medical device and each remote system as corresponding graphical icons on a display;
determine encryption levels of the plurality of communication links;
display connection graphics representing the plurality of communication links, each connection graphic is positioned between the medical device and one of the remote systems having a visual feature corresponding to an encryption level of a communication link between the medical device and the one of the remote systems.

19. The tangible and non-transitory computer readable medium of claim 18, wherein the one or more processors are further directed to receive a predetermined protected health information (PHI) workflow identifying a first remote system and a second remote system from the plurality of remote systems, wherein the medical device receives PHI from the first remote system and transmits an updated PHI to the second remote system.

20. The tangible and non-transitory computer readable medium of claim 19, wherein the one or more processors are further directed to add identification information of the second remote system and corresponding patient information from the updated PHI to a PHI transaction report when the medical device transmits the updated PHI.
